SEQUENCE LISTING (16 sequences)

SEQ ID NO: 1 - DNA, 20 bp, human
ctcctttcccaagcaagttg

SEQ ID NO: 2 - DNA, 20 bp, human
gggccatggaacaaggatga

SEQ ID NO: 3 - DNA, 20 bp, human
ccagccctcacctgcctagt

SEQ ID NO: 4 - DNA, 20 bp, human
caccgtgccactctcgttca

SEQ ID NO: 5 - DNA, 21 bp, human
agccctgccctgaagtcgtta

SEQ ID NO: 6 - DNA, 20 bp, human
cagggcatctcctgtgcatg

SEQ ID NO: 7 - DNA, 20 bp, human
ccaaggccgcatgactttga

SEQ ID NO: 8 - DNA, 21 bp, human
gccaagaaagggacatctatg

SEQ ID NO: 9 - DNA, 20 bp, human
tgttccggagcacggtcaag

SEQ ID NO: 10 - DNA, 18 bp, human
tgtaagcagctgaggtct

SEQ ID NO: 11 - DNA, 51 bp, artificial
  primer
aattctaatacgactcactatagggagaaggctcctttcccaagcaagttg

SEQ ID NO: 12 - DNA, 52 bp, artificial
  primer
aattctaatacgactcactatagggagaaggagccctgccctgaagtcgtta

SEQ ID NO: 13 - DNA, 22 bp, human
caggctgtcttgactgtcgtga

SEQ ID NO: 14 - DNA, 20 bp, human
aggagagaaaggatttggct

SEQ ID NO: 15 - DNA, 20 bp, human
gatccagggcggagacttca

SEQ ID NO: 16 - DNA, 53 bp, artificial
  primer
aattctaatacgactcactatagggagaaggcaggctgtcttgactgtcgtga

(19) United States
(12) Patent Application Publication
(10) Pub. No.: US 2008/0274465 A1
(43) Pub. Date: Nov. 6, 2008

(54) Method for Breast Cancer Diagnosis/Prognosis

(75) Inventor: Thibault Verjat, Lyon (FR)

Correspondence Address: OLIFF & BERRIDGE, PLC, P.O. BOX 320850, ALEXANDRIA, VA, 22320-4850, US

(73) Assignee: BIOMERIEUX, MARCY L'ETOILE (FR)

(21) Appl. No.: 11/919,923

(22) PCT Filed: Jun. 7, 2006
(86) PCT No.: PCT/FR2006/050531

§ 371 Date: Nov. 6, 2007

(30) Foreign Application Priority Data
Jun. 9, 2005 (FR) 0551548

(51) Int. Cl. C12Q 1/68; C07H 21/00; C12P 19/34

(52) U.S. Cl. 435/6; 536/24.1; 435/91.5; 536/24.3

(57) ABSTRACT

The present invention relates to a method for breast cancer diagnosis/prognosis comprising the following steps:
A—extracting the nucleic material from a biological sample;
B—using at least one pair of amplification primers to obtain amplicons of at least one target sequence of the nucleic material;
C—using at least one detection probe to detect the presence of said amplicons,
   characterized in that, during step B, said pair of primers comprises at least one amplification primer comprising at least 10 nucleotide motifs of a nucleotide sequence selected from SEQ ID No. 1 to SEQ ID No. 8 and/or, during step C, said detection probe comprises at least 10 nucleotide motifs of a nucleotide sequence selected from SEQ ID No. 9 and SEQ ID No. 10.

The invention also relates to amplification primers and hybridization probes which can be used in said method, and to a kit for the diagnosis/prognosis of breast cancer.

The present invention relates to a method for breast cancer diagnosis/prognosis. The invention also relates to amplification primers and hybridization probes which can be used in this method, and to a kit for the diagnosis/prognosis of breast cancer.

Breast cancer is a common disease: one woman in eleven develops breast cancer during her lifetime. However, because various types of breast cancer and various prognoses for breast cancer exist, women suffering from breast cancer do not all follow the same treatment: the physician proposes to each patient a treatment suitable for her situation, in order to obtain the best possible chances of recovery.

Thus, hormone therapy, which is a general treatment in breast cancer, is used in hormone-dependent breast cancers, i.e. in the case of tumors expressing hormone receptors at the surface of their cells. Postoperatively, hormone therapy can be used alone or as a switch from adjuvant chemotherapy. In the case of recurrence of the disease, hormone therapy can be prescribed either alone, or in combination with or as a switch from chemotherapy.

Chemotherapy is, for its part, a general cancer treatment since the medicaments, carried by the bloodstream, can act everywhere in the body. Chemotherapy has had an important place in the therapeutic arsenal, in particular over the past decade or so, with the appearance of new molecules. The medicaments are most commonly administered by intravenous infusion, by subcutaneous injection or by intramuscular injection.

Thus, the treatment will or will not be directed toward hormone therapy depending on the expression of hormone receptors at the surface of the hormonal cells.

Mention may in particular be made of estrogen receptors ERs and the progesterone receptor (PR), which are the most well known parameters for predicting the response to hormone therapy in breast cancer. Analysis of the expression of UPA (urokinase-type plasminogen activator) and PAI-1 (plasminogen activator inhibitor type 1) genes also makes it possible to benefit from a prognostic tool for breast cancer. These genes are involved in the destruction of the extracellular matrix and are therefore, as a result, factors involved in the metastatic development of tumors (Andreasen et al, Int. J. Cancer, 1997).

In order to provide patients with a suitable treatment, it is therefore essential to determine the expression of hormone receptors, such as ERs and PRs. This expression is most commonly studied on the primary tumor by immunohistochemistry. In addition, it is important to determine the expression of the HER2 receptor, the overexpression of which, studied by immunohistochemistry, is a factor for poor prognosis. In cases where there is a doubt, the study of a gene amplification of the HER2 gene by in-situ hybridization (FISH) is the alternative method used. Finally, analysis of the expression of UPA and PAI-1, which are tumor aggressiveness factors, is studied by ELISA (Enzyme-Linked Immunosorbent Assay).

For some years, it has been possible to detect small tumors, allowing an early diagnosis of breast cancer, but the prognosis for this cancer then remains difficult due to the small amount of tumor tissue which makes it difficult to carry out a protein quantification for the hormone receptors and factors mentioned above.

The present invention proposes a novel breast cancer diagnosis/prognosis method. This method employs in particular the analysis of the expression of the UPA and PAI-1 genes through the use of novel nucleotide sequences which can be used as amplification primers or hybridization probes. The method according to the invention makes it possible in particular to determine the prognosis for a patient suffering from breast cancer, in order to provide said patient with a suitable treatment.

In this respect, the invention relates to a method for breast cancer diagnosis/prognosis comprising the following steps:
A—extracting the nucleotide material from a biological sample;
B—using at least one pair of amplification primers to obtain amplicons of at least one target sequence of the nucleic material;
C—using at least one detection probe to detect the presence of said amplicons,
   characterized in that, during step B, said pair of primers comprises at least one amplification primer comprising at least 10 nucleotide motifs of a nucleotide sequence selected from SEQ ID No. 1 to SEQ ID No. 8 and/or, during step C, said detection probe comprises at least 10 nucleotide motifs of a nucleotide sequence selected from SEQ ID No. 9 and SEQ ID No. 10.

Surprisingly, the inventors have thus discovered that the use, in a method for breast cancer diagnosis/prognosis, of a nucleotide sequence comprising at least 10 nucleotide motifs of a nucleotide sequence selected from SEQ ID No. 1 to SEQ ID No. 8 is very relevant as an amplification primer for amplifying target sequences, such as the gene encoding UPA and PAI-1. The inventors have also discovered that the use of a nucleotide sequence comprising at least 10 nucleotide motifs of a nucleotide sequence selected from SEQ ID No. 9 and SEQ ID No. 10 as a hybridization probe is very relevant for specific hybridization on target sequences, such as the genes encoding UPA and PAI-1.

For the purposes of the present invention, the term “biological sample” is intended to mean any sample which may contain a nucleic material as defined hereinafter. This biological sample may be taken from a patient and may in particular be a tissue, blood, serum or saliva sample or sample of circulating cells from the patient. Preferably, this biological sample is taken from a tumor. This biological sample is obtained by any sampling means known to those skilled in the art.

For the purpose of the present invention, the term “nucleic material” includes a nucleic acid sequence such as a deoxyribonucleic acid (DNA) or ribonucleic acid (RNA) sequence. According to a preferred embodiment of the invention, the nucleic material includes a deoxyribonucleic acid sequence. According to a preferred embodiment of the invention, the nucleic material is extracted from a biological sample taken from a patient.

The term “nucleotide sequence” (or nucleic acid sequences, or nucleotide or oligonucleotide fragment, or polynucleotide) is intended to mean a series of nucleotide motifs assembled together by means of phosphoric ester bonds, characterized by the informational sequence of the natural nucleic acids, capable of hybridizing to another nucleic acid sequence, it being possible for the series to contain monomers of different structures and to be obtained from a natural nucleic acid molecule and/or by genetic recombination and/or by chemical synthesis.

The term “nucleotide motif” is intended to mean a derivative of a monomer which may be a natural nucleotide of a nucleic acid, the constitutive elements of which are a sugar, a phosphate group and a nitrogenous base; in DNA, the sugar is deoxy-2-ribose, in RNA, the sugar is ribose; depending on whether it is DNA or RNA, the nitrogenous base is selected from adenine, guanine, uracil, cytosine and thymine; or alternatively the monomer is a nucleotide modified in at least one of the three constitutive elements; by way of example, the modification may occur either at the level of the bases, with modified bases such as inosine, methyl-5-deoxycytidine, deoxyuridine, dimethylamino-5-deoxyuridine, diamino-2,6-purine, bromo-5-deoxyuridine or any other modified base capable of hybridization, or at the level of the sugar, for example replacement of at least one deoxyribose with a polyamide (P. E. Nielsen et al, Science, 254, 1497-1500 (1991)), or else at the level of the phosphate group, for example replacement of the latter with esters selected in particular from diphosphates, alkyl phosphonates, aryl phosphonates and phosphorothioates. This nucleic material comprises at least one target sequence. The term “target sequence” is intended to mean a sequence in which the series of nucleotide motifs is specific for a target gene, such as preferably the gene encoding UPA or PAI-1. According to a preferred embodiment of the invention, the target sequence is included in a gene selected from the genes encoding UPA or PAI-1. In the rest of the disclosure, the term “target sequence” will be used irrespective of whether it is single-stranded or double-stranded.

During step A the nucleic material is extracted from a biological sample by any protocol known to those skilled in the art. By way of indication, the nucleic acid extraction can be carried out by means of a step in which the cells present in the biological sample are lyzed in order to release the nucleic acids contained in the protein and/or lipid envelopes of the cells (such as cell debris which disturbs the subsequent reactions). By way of example, the methods of lysis as described in patent application WO 00/05338 on mixed magnetic and mechanical lysis, patent application WO 99/53304 on electrical lysis and patent application WO 99/15321 on mechanical lysis, can be used.

Those skilled in the art may use other well known methods of lysis, such as heat shock or osmotic shock, or chemical lyses with chaotropic agents such as guanidinium salts (U.S. Pat. No. 5,234,809). This lysis step may also be followed by a purification step, allowing the nucleic acids to be separated from the other cellular constituents released during the lysis step. This step generally makes it possible to concentrate the nucleic acids, and can be adapted for the purification of DNA or of RNA. By way of example, it is possible to use magnetic particles optionally coated with oligonucleotides, by adsorption or covalence (in this respect, see patents U.S. Pat. No. 4,672,040 and U.S. Pat. No. 5,750,338), and thus to purify the nucleic acids which are bound to these magnetic particles, by means of a washing step. This nucleic acid purification step is particularly advantageous if it is desired to subsequently amplify said nucleic acids. A particularly advantageous embodiment of these magnetic particles is described in patent applications WO 97/45202 and WO 99/35500. Another advantageous example of a nucleic acid purification method is the use of silica, either in the form of a column or in the form of inert particles (Boom R. et al., J. Clin. Microbiol., 1990, n^{o}28 (3), p. 495-503) or magnetic particles (Merck: MagPrep® Silica, Promega: MagneSil™ Paramagnetic particles). Other very widely used methods are based on ion exchange resins in a column or in a paramagnetic particulate format (Whatman: DEAE-Magarose) (Levison P R et al., J. Chromatography, 1998, p. 337-344). Another method, which is very relevant but not exclusive to the invention, is that of adsorption onto a metal oxide support (company Xtrana: Xtra-Bind™ matrix).

When it is desired to specifically extract the DNA from a biological sample, an extraction with phenol, chloroform, and alcohol can in particular be carried out in order to remove the proteins, and the DNA can be precipitated with 100% alcohol. The DNA can then be pelleted by centrifugation, washed and resuspended.

During step B, at least one pair of amplification primers is used to obtain amplicons of at least one target sequence of the nucleic material.

For the purposes of the present invention, the term “amplification primer” is intended to mean a nucleic sequence comprising from 10 to 100 nucleotide motifs, preferably from 15 to 25 nucleotide motifs. This amplification primer comprises at least 10, preferably 15, and even more preferably 20 nucleotide motifs of the sequence selected from SEQ ID Nos. 1 to 8.

A pair of amplification primers makes it possible to initiate an enzymatic polymerization, such as in particular an enzymatic amplification reaction.

The term “enzymatic amplification reaction” is intended to mean a process which generates multiple copies (or amplicons) of a nucleic sequence through the action of at least one enzyme. For the purposes of the present invention, the term “amplicons” is intended to mean the copies of the target sequence that are obtained during an enzymatic amplification reaction. Such amplification reactions are well known to those skilled in the art and mention may in particular be made of PCR (polymerase chain reaction), as described in patents U.S. Pat. No. 4,683,195, U.S. Pat. No. 4,683,202 and U.S. Pat. No. 4,800,159; LCR (ligase chain reaction), disclosed, for example, in patent application EP-A-0 201 184; RCR (repair chain reaction), described in patent application WO-A-90/01069; 3SR (self-sustained sequence replication) with patent application WO-A-90/06995; NASBA (nucleic acid sequence-based amplification) with patent application WO-A-91/02818, or else TMA (transcription-mediated amplification) with patent U.S. Pat. No. 5,399,491.

In general, these enzymatic amplification reactions generally implement a succession of cycles comprising the following steps:
denaturation of the target sequence if the latter is double-stranded, in order to obtain two target strands which are complementary,
hybridization of each of the target strands, obtained during the preceding denaturation step, with at least one amplification primer,
formation, from the amplification primers, of the strands complementary to the strands on which they are hybridized, in the presence of a polymerase enzyme and of nucleoside triphosphates (ribonucleoside triphosphate and/or deoxyribonucleoside trisphosphate depending on techniques),
   this cycle being repeated a given number of times in order to obtain the target sequence in a proportion sufficient to allow its detection.

The term “hybridization” is intended to mean the process during which, under suitable conditions, two nucleic sequences, such as, in particular, an amplification primer and a target sequence or a hybridization probe and a target sequence, bond to one another with stable and specific hydrogen bonds so as to form a double strand. These hydrogen bonds form between the complementary bases adenine (A) and thymine (T) (or uracil (U)) (this is then referred to as an A-T bond) or between the complementary bases guanine (G) and cytosine (C) (this is then referred to as a G—C bond). The hybridization of two nucleic sequences may be total (reference is then made to complementary sequences), i.e. the double strand obtained during this hybridization comprises only A-T bonds and C-G bonds. This hybridization may be partial (reference is then made to sufficiently complementary sequences), i.e. the double strand obtained comprises A-T bonds and C-G bonds allowing the double strand to form, but also bases not bonded to a complementary base. The hybridization between two complementary sequences or sufficiently complementary sequences depends on the operating conditions that are used, and in particular the stringency. The stringency is defined in particular according to the base composition of the two nucleic sequences, and also by the degree of mismatching between these two nucleic sequences. The stringency can also depend on the reaction parameters, such as the concentration and the type of ionic species present in the hybridization solution, the nature and the concentration of denaturing agents and/or the hybridization temperature. All these data are well known and the appropriate conditions can be determined by those skilled in the art.

More specifically, NASBA is a technology consisting of isothermal amplification of the nucleic acid, based on the joint action of three enzymes (AMV reverse transcriptase, Rnase-H and T7 RNA polymerase). Combined with amplification primers specific for a target sequence, it amplifies the RNA targets more than a billion times in 90 minutes. The amplification reaction is carried out at 41° C. and gives single-stranded RNA molecules as the final product. NASBA requires a pair of primers, at least one of which comprises a promoter for the initiation of transcription with a T7 bacteriophage polymerase. Such a primer is preferably selected from SEQ ID Nos. 11 and 12. According to a specific embodiment of the invention, said pair of primers comprises at least one amplification primer comprising a promoter for the initiation of transcription with a T7 bacteriophage polymerase.

According to a specific embodiment of the invention, said pair of primers, used in step B, is selected from the following pairs of primers:
a first amplification primer comprising at least 10, preferably 15, and even more preferably 20 nucleotide motifs of the nucleotide sequence SEQ ID No. 1 and a second amplification primer comprising at least 10, preferably 15, and even more preferably 20 nucleotide motifs of the nucleotide sequence SEQ ID No. 2; by way of indication, when the first primer has the sequence SEQ ID No. 1 and the second primer has the sequence SEQ ID No. 2, an amplicon which is specific for the gene encoding PAI-1, which is 216 base pairs in size, and which corresponds to sequence 394-610 on the reference sequence of the gene encoding PAI-1 (Genbank NM_{—}000602.1), is obtained;
a first amplification primer comprising at least 10, preferably 15, and even more preferably 20 nucleotide motifs of the nucleotide sequence SEQ ID No. 3 and a second amplification primer comprising at least 10, preferably 15, and even more preferably 20 nucleotide motifs of the nucleotide sequence SEQ ID No. 4; by way of indication, when the first primer has the sequence SEQ ID No. 3 and the second primer has the sequence SEQ ID No. 4, an amplicon which is specific for the gene encoding PAI-1, which is 1058 base pairs in size, and which corresponds to sequence 88-1146 on the reference sequence encoding PAI-1 (Genbank NM_{—}000602.1), is then obtained;
a first amplification primer comprising at least 10, preferably 15, even more preferably 20 nucleotide motifs of the nucleotide sequence SEQ ID No. 5 and a second amplification primer comprising at least 10, preferably 15, and even more preferably 20 nucleotide motifs of the nucleotide sequence SEQ ID No. 6; by way of indication, when the first primer has the sequence SEQ ID No. 5 and the second primer has the sequence SEQ ID No. 6, an amplicon which is specific for the UPA gene, which is 194 base pairs in size, and which corresponds to sequence 1661-1855 on the reference sequence encoding UPA (Genbank NM_{—}002658.1), is then obtained;
a first amplification sequence comprising at least 10, preferably 15, and even more preferably 20 nucleotide motifs of the nucleotide sequence SEQ ID No. 7 and a second amplification primer comprising at least 10, preferably 15, and even more preferably 20 nucleotide motifs of the nucleotide sequence SEQ ID No. 8; by way of indication, when the first primer has a sequence SEQ ID No. 7 and the second primer has the sequence SEQ ID No. 8, an amplicon which is specific for the gene encoding UPA, which is 927 base pairs in size, and which corresponds to sequence 1228-2155 on the reference sequence encoding UPA (Genbank NM_{—}002658.1), is then obtained.

According to a specific embodiment of the invention, said pair of primers, used in step B, comprises a first primer comprising a promoter for the initiation of transcription with a T7 bacteriophage polymerase, and is selected from the following pairs of primers:
a first amplification primer of SEQ ID No. 11 and a second amplification primer comprising at least 10, preferably 15, and even more preferably 20 nucleotide motifs of the nucleotide sequence SEQ ID No. 2;
a first amplification primer of SEQ ID No. 12 and a second amplification primer comprising at least 10, preferably 15, and even more preferably 20 nucleotide motifs of the nucleotide sequence SEQ ID No. 6.

In order to take into account the variability in enzymatic efficiency that may be observed during the various steps of the amplification reaction, the expression of a target gene can be standardized by the simultaneous determination of the expression of a “housekeeping” gene, the expression of which is similar in the various groups of patients. By effecting a ratio between the expression of the target gene and the expression of the housekeeping gene, any varability between the various experiments is thus corrected. Those skilled in the art now refer in particular to the following publications: Bustin S A Journal of molecular endocrinology, 2002, 29: 23-39; Giulietti A Methods, 2001, 25: 386-401. According to a specific embodiment of the invention, during step B, use is also made of at least one pair of amplification primers to obtain amplicons specific for a housekeeping gene. The term “housekeeping gene” is intended to mean a gene whose expression is stable in a given tissue, irrespective of the physiological situation. According to a preferred embodiment of the invention, the housekeeping gene is the PPIB gene which encodes cyclophylin B. Those skilled in the art also refer in particular to application PCT/FR04/050661 which is included in the present application by way of reference.

During step C, at least one detection probe is used to detect the presence of said amplicons. This detection step can be carried out by any of the protocols known to those skilled in the art concerning nucleic acid detection.

For the purpose of the present invention, the term “hybridization probe” is intended to mean a nucleic sequence of 10 to 100 nucleotide motifs, in particular of 15 to 35 nucleotide motifs, having a hybridization specificity under given conditions so as to form a hybridization complex with a target nucleic sequence. The hybridization probe can comprise a label for its detection. Reference is then made to detection probes. The term “detection” is intended to mean either a direct detection by a physical method, or an indirect detection by a method of detection using a label. Many methods of detection exist for the detection of nucleic acids [see, for example, Kricka et al., Clinical Chemistry, 1999, n^{o} 45 (4), p. 453-458 or Keller G. H. et al., DNA Probes, 2nd Ed., Stockton Press, 1993, sections 5 and 6, p. 173-249]. The term “label” is intended to mean a tracer capable of generating a signal that can be detected. A nonlimiting list of these tracers includes enzymes which produce a signal that is detectable, for example by colorimetry, fluorescence or luminescence, such as horseradish peroxidase, alkaline phosphatase, beta-galactosidase, glucose-6-phosphate dehydrogenase; chromophores, such as fluorescent, luminescent or dye compounds; electron-dense groups that can be detected by electron microscopy or by their electrical properties such as conductivity, by amperometry or voltametry methods, or by impedance measurements; groups that can be detected by optical methods such as diffraction, surface plasmon resonance, contact angle variation or by physical methods such as atomic force spectroscopy, tunnel effect, etc.; radioactive molecules such as ³²P, ³⁵S or ¹²⁵I.

For the purpose of the present invention, the hybridization probe may be a “detection” probe. In this case, the “detection” probe is labeled by means of a label as defined above. By virtue of the presence of this label, it is possible to detect the presence of a hybridization reaction between a given detection probe and the specific target sequence of a given species.

The detection probe may in particular be a “molecular beacon” detection probe as described by Tyagi & Kramer (Nature biotech, 1996, 14:303-308). These “molecular beacons” become fluorescent during hybridization. They have a stem-loop structure and contain a fluorophore and a quencher group. The binding of the specific loop sequence with its complementary target nucleic acid sequence causes the stem to uncoil and a fluorescent signal to be emitted during excitation at the appropriate wavelength.

The hybridization probe may also be a capture probe. In this case, the “capture” probe is immobilized or can be immobilized on a solid support by any appropriate means, i.e. directly or indirectly, for example by covalence or adsorption. The hybridization reaction between a given capture probe and a target sequence is then detected.

For the detection of the hybridization reaction, use may be made of target sequences that have been labeled, directly (in particular by the incorporation of a label within the target sequence) or indirectly (in particular using a detection probe as defined above) the target sequence. A step for labeling and/or cleaving the target sequence can in particular be carried out before the hybridization step, for example using a labeled deoxyribonucleotide triphosphate during the enzymatic amplification reaction. The cleavage can be carried out in particular through the action of imidazole and manganese chloride. The target sequence can also be labeled after the amplification step, for example by hybridizing a detection probe according to the sandwich hybridization technique described in document WO 91/19812. Another specific preferred method for labeling nucleic acids is described in application FR 2 780 059.

As solid support, use may be made of synthetic materials or natural materials, optionally chemically modified, in particular polysaccharides, such as cellulose-based materials, for example paper, cellulose derivatives such as cellulose acetate and nitrocellulose, or dextran, polymers, copolymers, in particular based on styrene-type monomers, natural fibers such as cotton, and synthetic fibers such as nylon; mineral materials such as silica, quartz, glasses, ceramics; latices; magnetic particles; metal derivatives, gels, etc. The solid support can be in the form of a microtitration plate, of a membrane as described in application WO 94/12670, or of a particle.

According to a preferred embodiment of the invention, the detection probe comprises a fluorophore and a quencher. According to an even more preferred embodiment of the invention, the hybridization probe comprises a FAM (6-carboxyfluorescein) or a ROX (6-carboxy-X-rhodamine) fluorophore at its 5′ end and a quencher (Dabsyl) at its 3′ end. In the subsequent disclosure, such a hybridization probe is known as “molecular beacon”.

According to a preferred embodiment of the invention, steps B and C are carried out at the same time. This preferred method can be carried out by “realtime NASBA”, which combines, in a single step, the NASBA amplification technique and realtime detection using molecular beacons. The NASBA reaction is carried out in the tube, producing the single-stranded RNA with which the specific molecular beacons can simultaneously hybridize to give a fluorescent signal. The formation of the new RNA molecules is measured in realtime by continuous monitoring of the signal in a fluorescent reader. Unlike an amplification by RT-PCR, amplification by NASBA can be carried out in the presence of DNA in the sample, i.e. even if the DNA has not been completely removed during the RNA extraction.

As presented in the example hereinafter, when it is desired to detect the target gene encoding PAI-1 (reference sequence NCBI accession number: NM_{—}000602.1), use is preferably made, during step b), of
a first primer of SEQ ID No. 1 or 11
a second primer of SEQ ID No. 2
   and, during step c), of
a detection probe comprising SEQ ID No. 9.

As presented in the example hereinafter, when it is desired to detect the target gene encoding UPA (reference sequence NCBI accession number: NM_{—}002658.1), use is preferably made, during step b), of
a first primer of SEQ ID No. 5 or 12
a second primer of SEQ ID No. 6
   and, during step c), of
a detection probe comprising SEQ ID No. 10.

When, during step B, a pair of amplification primers is used to obtain amplicons specific for a housekeeping gene, said amplicons specific for a housekeeping gene can be detected in a manner comparable to that described above, in particular using a detection probe. According to a preferred embodiment of the invention, the housekeeping gene is the PPIB gene which encodes cyclophilin B. Preferably, the detection probe comprises a fluorophore and a quencher.

The invention also relates to an amplification primer comprising at least 10, preferably 15, and even more preferably 20 nucleotide motifs of a nucleotide sequence selected from SEQ ID No. 1 to SEQ ID No. 8.

According to a preferred embodiment of the invention, the amplification primer comprises a promoter for the initiation of transcription with a T7 bacteriophage polymerase. This primer can in particular be either one of SEQ ID Nos. 11 and 12, and is preferably used in a NASBA amplification reaction.

The invention also relates to a pair of primers selected from the following pairs of primers:
a first amplification primer comprising at least 10, preferably 15, and even more preferably 20 nucleotide motifs of the nucleotide sequence SEQ ID No. 1 and a second amplification primer comprising at least 10, preferably 15, and even more preferably 20 nucleotide motifs of the nucleotide sequence SEQ ID No. 2; by way of indication, when the first primer has the sequence SEQ ID No. 1 and the second primer has the sequence SEQ ID No. 2, an amplicon which is specific for the gene encoding PAI-1, which is 216 base pairs in size, and which corresponds to sequence 394-610 on the reference sequence of the gene encoding PAI-1 (Genbank NM_{—}000602.1), is obtained;
a first amplification primer comprising at least 10, preferably 15, and even more preferably 20 nucleotide motifs of the nucleotide sequence SEQ ID No. 3 and a second amplification primer comprising at least 10, preferably 15, and even more preferably 20 nucleotide motifs of the nucleotide sequence SEQ ID No. 4; by way of indication, when the first primer has the sequence SEQ ID No. 3 and the second primer has the sequence SEQ ID No. 4, an amplicon which is specific for the gene encoding PAI-1, which is 1058 base pairs in size, and which corresponds to sequence 88-1146 on the reference sequence of the gene encoding PAI-1 (Genbank NM_{—}000602.1), is obtained;
a first amplification primer comprising at least 10, preferably 15, and even more preferably 20 nucleotide motifs of the nucleotide sequence SEQ ID No. 5 and a second amplification primer comprising at least 10, preferably 15, and even more preferably 20 nucleotide motifs of the nucleotide sequence SEQ ID No. 6; by way of indication, when the first primer has the sequence SEQ ID No. 5 and the second primer has the sequence SEQ ID No. 6, an amplicon which is specific for the UPA gene, which is 194 base pairs in size, and which corresponds to sequence 1661-1855 on the reference sequence encoding UPA (Genbank NM_{—}002658.1), is then obtained;
a first amplification primer comprising at least 10, preferably 15, and even more preferably 20 nucleotide motifs of the nucleotide sequence SEQ ID No. 7 and a second amplification primer comprising at least 10, preferably 15, and even more preferably 20 nucleotide motifs of the nucleotide sequence SEQ ID No. 8; by way of indication, when the first primer has the sequence SEQ ID No. 7 and the second primer has the sequence SEQ ID No. 8, an amplicon which is specific for the UPA gene, which is 927 base pairs in size, and which corresponds to sequence 1228-2155 on the reference sequence encoding UPA (Genbank NM_{—}002658.1), is then obtained.

According to a preferred embodiment of the invention, said first primer comprises a promoter for the initiation of transcription with a T7 bacteriophage polymerase. This primer can in particular be either one of SEQ ID Nos. 11 and 12. When the first primer comprises a promoter for the initiation of transcription with a T7 bacteriophage polymerase, this first primer is preferably included in a pair of primers selected from the following pairs of primers:
a first amplification primer of SEQ ID No. 21 and a second amplification primer comprising at least 10, preferably 15, and even more preferably 20 nucleotide motifs of the nucleotide sequence SEQ ID No. 2;
a first amplification primer of SEQ ID No. 22 and a second amplification primer comprising at least 10, preferably 15, and even more preferably 20 nucleotide motifs of the nucleotide sequence SEQ ID No. 4.

The invention also relates to the use of at least one amplification primer as defined above and/or of at least one pair of primers as defined above, in a NASBA amplification reaction.

The invention also relates to a detection probe comprising at least 10, preferably 15, and even more preferably 20 nucleotide motifs of a nucleotide sequence selected from SEQ ID No. 9 and SEQ ID No. 10.

Preferably, this detection probe comprises a fluorophore and a quencher.

The invention also relates to the use of at least one primer as defined above and/or of at least one pair of primers as defined above and/or of at least one detection probe as defined above, for the diagnosis/prognosis of breast cancer.

Finally, the invention relates to a kit for the diagnosis/prognosis of breast cancer, comprising at least one primer as defined above and/or at least one pair of primers as defined above and/or at least one detection probe as defined above.

As presented in the example hereinafter, when it is desired to detect the target gene encoding PAI-1, the kit preferably comprises:
a first primer of SEQ ID No. 1 or 11
a second primer of SEQ ID No. 2
a detection probe comprising SEQ ID No. 9.

As presented in the example hereinafter, when it is desired to detect the target gene encoding UPA, the kit preferably comprises:
a first primer of SEQ ID No. 5 or 12
a second primer of SEQ ID No. 6
a detection probe comprising SEQ ID No. 10.

The invention also relates to a kit for the diagnosis/prognosis of breast cancer, which makes it possible to determine
whether a patient suffering from breast cancer may benefit from a hormone therapy (tamoxifen) or from an immunotherapy (herceptin),
whether the cancer in question has a good or poor prognosis,
the aggressiveness of the tumor.

For this, the kit comprises the necessary reagents, such as primers and/or probes, for detecting the ER, PR, HER-2, UPA and/or PAI-1 genes. For the primers and probes necessary for the detection of the ER, PR, and HER-2 genes, those skilled in the art will refer in particular to patent application PCT/FR04/050661, which is included by way of reference. For the primers and probes necessary for the detection of the UPA and PAI-1 genes, those skilled in the art will refer to the disclosure developed above.

The following FIGURE is given by way of illustration and is in no way limiting in nature. It will obviously make it possible to understand the invention more clearly.

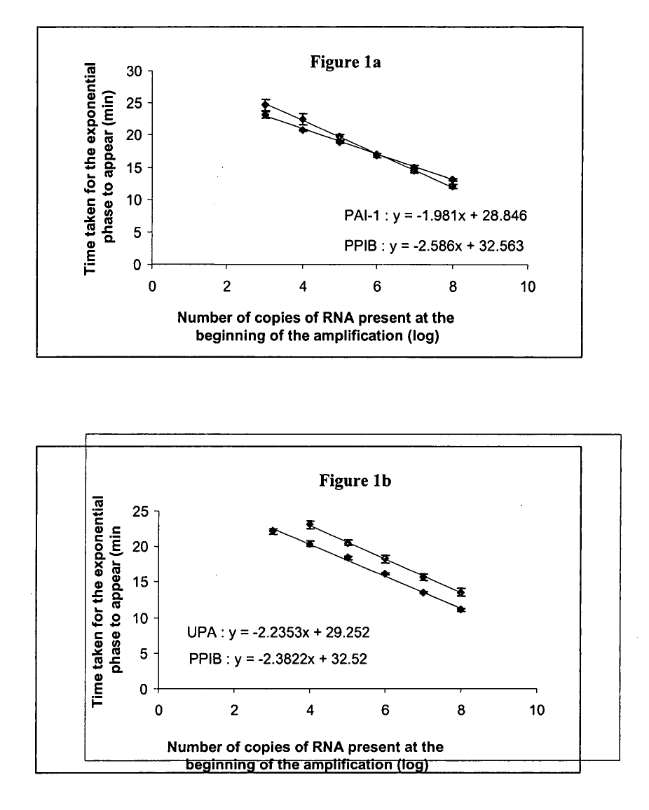

FIG. 1 represents the standard curves obtained for the PAI-1 and PPIB genes (FIG. 1a, PAI-1 as a solid line, PPIB as a dashed line), and the UPA and PPIB genes (FIG. 1b, UPA as a solid line, PPIB as a dashed line), as described in the example below. Each standard curve, obtained for each gene based on a reference sequence which is specific for the gene, transcribed into RNA in vitro using the PCR product specific for the UPA and PAI-1 gene, respectively, and a plasmid for the PPIB gene, represents the time taken for the exponential phase of amplification to appear (also referred to as TTP: time to positivity, threshold time) as a function of the number of copies of RNA present at the beginning of the amplification by NASBA (the greater the starting amount of RNA copies at the beginning of amplification, the shorter the time to positivity).

The following examples are given by way of illustration and are in no way limiting in nature. They will make it possible to understand the invention more clearly.

### EXAMPLE 1

### Amplification and Realtime Detection of mRNAs Encoding PAI-1 and UPA

1/Obtaining and Preparing Samples

This example was carried out using three tumor cell lines, in which the expression of the UPA and PAI-1 proteins has previously been determined by ELISA; the following were used: MDA-MD-231 (expressing the factors UPA and PAI-1), and MCF7 (expressing neither UPA nor PAI-1). These lines originate from the American Type Culture Collection (ATCC, Manassas, USA). These cell lines were cultured in a DMEM medium (MCF-7) or Leibovitz medium (MDA-MD-231), supplemented with fetal bovine serum (10%), nonessential amino acids (1%) and penicillin (50 000 U)-streptomycin (50 μg), at 37° C. under an atmosphere containing 5% CO₂ for MCF7 and 0% MDA-MD-231.

This example was also carried out using tumors from patients (n=77) suffering from a breast cancer in which the expression of UPA and PAI-1 was determined beforehand by ELISA according to a conventional technique known to those skilled in the art. The ELISA technique revealed the expression of the protein.

2/Total RNA Extraction

The total RNA was extracted from cell lines using Trizol® Reagent according to the recommendations of the kit supplier (Invitrogen, Canada). The quality and quantity of RNA were determined at 260 to 280 nm and verified on an agarose gel. The RNA was then frozen at −70° C. until use.

Total RNA was also extracted in a comparable manner from tumors of patients suffering from breast cancer.

3) Amplification by NASBA

The NASBA amplification reaction is based on the simultaneous activity of a reverse transcriptase of the avian myeloblast virus (AMV-RT), of an E. coli RNAse H and of a T7 bacteriophage RNA polymerase (Compton J, 1991, Nature, 350: 91-92). The realtime detection of the amplicons is carried out using a Nuclisens EasyQ® reader (bioMérieux BV, The Netherlands) and “molecular beacon” detection probes, as defined above. The NASBA quantification is based on the use of a standard curve, obtained from a reference sequence, specific for the target gene, transcribed into RNA in vitro in a plasmid. This standard curve represents the time to positivity as a function of the number of RNA copies present at the beginning of the NASBA amplification (the greater the amount of RNA copies at the beginning of the amplification, the shorter the time to positivity).

### a) Amplification of the PAI-1 and UPA Genes and of the PPIB Housekeeping Gene—Obtaining of a Standard Curve

Standard Curve for the Target Gene Encoding PAI-1 (FIG. 1a)

For the target gene encoding PAI-1 (reference sequence NCBI accession number: NM_{—}000602.1), use was made of a first primer of SEQ ID No. 3, 5′ CCAGCCCTCA CCTGCCTAGT 3′, having an additional sequence corresponding to the SP6 promoter, and a second Primer of SEQ ID No. 4, 5′ CACCGTGCCA CTCTCGTTCA 3′, located respectively at position 88-107 and 1127-1146 of the reference sequence, in order to generate by PCR (a 1^{st} denaturation cycle (95° C.; 1 min), then 35 cycles comprising the following steps: denaturation: 94° C.; 1 min; hybridization: 60° C.; 1 min; elongation: 72° C.; 2 min, and a final cycle comprising a denaturation step: 72° C.; 7 min) an amplicon of 1058+23 (of the SP6 sequence) base pairs, specific for the gene encoding PAI-1 (this will be referred to as “PAI-1 amplicon”).

The PAI-1 amplicons obtained as described above were then purified (Qiaquick® column, Qiagen, Hilden, Germany) and then transcribed directly into RNA in vitro using an RNA polymerase (SP6 (Megascript® kit, Ambion, Austin, USA), according to the orientation of the amplicon). After elimination of the plasmid by treatment with DNAse, the RNAs were purified using the Rneasy® mini kit (Qiagen, Hilden, Germany) and quantified (RNA6000 Nano, Agilent Technologies, Walbronn, Germany). The amplicons obtained ere indeed specific for the PAI-1 gene.

The RNAs obtained above were diluted to various concentrations (stock solution: 0.2×10¹¹ copies/μl, cascade dilution from 0.2×10¹¹ copies/μl to 0.2×10² copies/μl). These cascade dilutions were amplified by NASBA using the Nuclisens Basic® kit (bioMérieux BV, The Netherlands) in the presence of the specific PAI-1 primer SEQ ID No. 1 and specific PAI-1 primer SEQ ID No. 2, and of the “molecular beacon” SEQ ID No. 9:
0.2 μM of a first PAI-1 primer of SEQ ID No. 1, 5′ CTCCTTTCCC AAGCAAGTTG 3′, comprising, at its 5′ end, and indicated in lower case, a sequence comprising the promoter of the T7 polymerase, i.e. a first primer of which the whole sequence is SEQ ID No. 11: 5′ aattctaatacgactcactatagggagaaggCTCCTTTCCCAAGCAAGTTG 3′;
0.2 μM of a second PAI-1 primer of SEQ ID No. 2, 5′ GGGCCATGGA ACAAGGATGA 3′;
0.1 μM of “molecular beacons” comprising SEQ ID No. 9 5′ 3′, labeled with a FAM (6-carboxyfluorescein) fluorophore in the 5′ position and a quencher (Dabsyl) in the 3′ position (complete sequence: 5′ TGTTCCGGAG CACGGTCAAG 3′. FAM-cgatcgTGTTCCGGAGCACGGTCAAGcgatcg-Dabsyl).

During the amplification, the signal intensifies proportionally to the amount of amplicons produced. The curve of fluorescence as a function of time makes it possible to define the time where the exponential phase of amplification will begin (also called TTP: time to positivity, threshold time). The PAI-1 standard curve links the number of transcripts present in the solution at the start as a function of the TTP detected during the NASBA amplification. Using a standard curve, the absolute number of copies of the target gene is calculated. Finally, this value is normalized by virtue of a housekeeping gene, in this case the PPIB gene. This PAI-1 standard curve is represented in FIG. 1a (solid-line curve).

Standard Curve for the Target Gene Encoding UPA

The curve for the gene encoding UPA was produced according to the same principle as for PAI-1, with the exception of the amplification primers used and the molecular beacons, which were specific for UPA.

Thus, for the gene encoding UPA (reference sequence NCBI accession number: NM_{—}002658.1), use was made of a first primer of SEQ ID No. 7, 5′ CCAAGGCCGC ATGACTTTGA 3′, and a second of SEQ ID No. 8, 5′ GCCAAGAAAG GGACATCTATG 3′, located respectively at position 1228-1248 and 2135-2155 of the reference sequence.

The amplicons were then transcribed into RNA in vitro using an RNA polymerase (T7 or SP6 (Megascript® kit, Ambion, Austin, USA), according to the orientation of the amplicon). After elimination of the plasmid by treatment with DNAse, the RNAs were purified by means of an Rneasy® mini kit (Qiagen, Hilden, Germany) and quantified (RNA6000 Nano, Agilent Technologies, Walbronn, Germany). The amplicons obtained were indeed specific for the PAI-1 gene.

The RNAs obtained above were diluted to various concentrations (stock solution: 0.2×10¹¹ copies/μl, cascade dilution from 0.2×10¹¹ copies/μl to 0.2×10² copies/μl). These cascade dilutions were amplified by NASBA using the Nuclisens Basic® kit (bioMérieux BV, The Netherlands) in the presence of
0.2 μM of a first UPA primer of SEQ ID No. 5, 5′ AGCCCTGCCC TGAAGTCGTT A 3′, comprising, at its 5′ end, and indicated in lower case, a sequence comprising the promoter of the T7 polymerase, i.e. a first primer for which the whole sequence is SEQ ID No. 12: 5′ aattctaatacgactcactatagggagaaggAGCCCTGCCCTGAAGTCGTTA 3′,
0.2 μM of a second UPA primer of SEQ ID No. 6, 5′ CAGGGCATCT CCTGTGCATG 3′,
0.1 μM of molecular beacons used, comprising SEQ ID No. 10, 5′ TGTAAGCAGC TGAGGTCT 3′, labeled with a FAM (6-carboxyfluorescein) fluorophore at their 5′ end and with a quencher (Dabsyl) at their 3′ end (complete sequence: 5′ FAM-cgatcg TGTAAGCAGC TGAGGTCT cgatcg-Dabsyl 3′).

The UPA standard curve is represented in FIG. 1b.

Standard Curve for the PPIB Target Gene

The curve for the PPIB target gene was produced according to the same principle as for PAI-1, with the exception of the amplification primers and of the molecular beacons, which were specific for PPIB.

Thus, for the PPIB housekeeping gene (reference sequence NCBI accession number: M60857), use was made of a first primer of SEQ ID No. 13, 5′ ACATGAAGGT GCTCCTTGCC 3′, and a second primer of SEQ ID No. 14, 5′ GTCCCTGTGC CCTACTCCTT 3′, located respectively at positions 11-30 and 631-650 of the reference sequence. The sequence of these PPIB amplicons was verified by sequencing (Biofidal, Vaulx en Velin, France), in order to be sure that it indeed corresponded to the sequence of the target gene that was intended to be amplified. The amplicons obtained were indeed specific for the PPIB gene.

The amplicons were then transcribed to RNA in vitro using an RNA polymerase (T7 or SP6 (Megascript® kit, Ambion, Austin, USA), according to the orientation of the amplicon. After elimination of the plasmid by treatment with DNAse, the RNAs were purified by means of an Rneasy® mini kit (Qiagen, Hilden, Germany) and quantified (RNA6000 Nano, Agilent Technologies, Walbronn, Germany).

The RNAs obtained above were diluted to various concentrations (stock solution: 0.2×10¹¹ copies/μl, cascade dilution from 0.2×10¹¹ copies/μl to 0.2×10² copies/μl). These cascade dilutions were amplified by NASBA using the Nuclisens Basic® kit (bioMérieux BV, The Netherlands) in the presence of:
0.2 μM of a first PPIB primer of SEQ ID No. 13, 5′ CAGGCTGTCT TGACTGTCGT GA 3′, comprising, at its 5′ end, and indicated in lower case, a sequence comprising the promoter of the T7 polymerase, i.e. a first primer of which the whole sequence is SEQ ID No. 16: 5′ aattctaata cgactcacta tagggagaag gCAGGCTGTCTTGACTGTCG TGA 3′;
0.2 μM of a second PPIB primer of SEQ ID No. 4, 5′ AGGAGAGAAA GGATTTGGCT 3′;
0.1 μM of molecular beacons comprising SEQ ID No. 15, 5′ GATCCAGGGCGGAGACTTCA 3′, labeled with a ROX (6-carboxy-X-rhodamine) fluorophore in the 5′ position and a quencher (Dabsyl) in the 3′ position (complete sequence: 5′ ROX-cgatcgGATCCAGGGCGGAGACTTCAcga cg-Dabsyl 3′).

The PPIB standard curve is represented in FIGS. 1A and 1B (dashed-line curves).

### b) NASBA Amplification Reaction:

b1) Duplex Amplification of the PAI-1 and PPIB Genes

This amplification reaction was carried out using a Nuclisens Basic® kit (bioMérieux BV, The Netherlands). For this, 5 ng of total RNA, extracted from the various cell lines, were added to 10 μl of NASBA buffer (final concentration in 20 μl of reaction medium: 40 mM of tris HCl, pH 8.5, 12 mM MgCl₂, 70 mM KCl, 5 mM dithiotreitol, 15% v/v DMSO, 1 mM of each dNTP, 2 mM of each NTP).

Added to this medium was 0.1 μM of molecular beacons comprising:
SEQ ID No. 9, labeled with a FAM (6-carboxyfluorescein) fluorophore in the 5′ position and a quencher (Dabsyl) in the 3′ position, for detecting RNA of the PAI-1 gene;
SEQ ID No. 15, labeled with a ROX (6-carboxy-X-rhodamine) fluorophore in the 5′ position and a quencher (Dabsyl) in the 3′ position (for detecting the RNA of the PPIB gene).

Also added to this medium were:
0.2 μM of a first PAI-1 primer of SEQ ID No. 1, comprising, at its 5′ end, a sequence comprising the promoter of the T7 polymerase,
0.2 μM of a second PAI-1 primer of SEQ ID No. 2,
0.2 μM of a first PPIB primer of SEQ ID No. 13, comprising, at its 5′ end, a sequence comprising the promoter of the T7 polymerase,
0.2 μM of a second PPIB primer of SEQ ID No. 14.

A preincubation was carried out for 2 minutes at 65° C. before an incubation of 2 minutes at 41° C. A volume of 5 μl of an enzyme mix (0.08 U of RNAse H, 32 U of T7 RNA polymerase, 6.4 U of AMV-RT) was added, and an incubation of 90 minutes at 41° C. was carried out.

The quantification of the RNAs transcribed was determined in real time (NucliSens EasyQ, bioMérieux), the NASBA reaction producing amplicons with which the specific molecular beacons can hybridize simultaneously so as to give a fluorescent signal. The formation of the new RNA molecules is measured in real time by continuous monitoring of the signal in a fluorescence reader, the NucliSens EasyQ analyzer. The data analysis and automated communication are provided by the Nuclisens TTP software (bioMérieux BV, The Netherlands). The standard curve, as defined above (dilution transcribed RNA: 10⁸ to 10² copies) was used to quantify the expression of each of the ESR1 target gene and of the PPIB housekeeping gene, in order to extrapolate the number of copies of mRNA per sample. The quantification of the expression of a target gene was expressed by number of copies of mRNA/5 ng of total RNA.

Table 1 gives the expression of the PAI-1 gene quantified using 5 ng of total RNA originating from the MDA-MD-231 and MCF7 cell lines.

TABLE 1
Expression of the PAI-1 gene in MCF7 and T47D cells
(NC: not calculable)

| | Number of | Number of | |
|---|---|---|---|
| | copies of PAI-1 | copies of PPIB | |
| | mRNA | mRNA | PAI-1/PPIB |
| MDA-MD-231 | 2.52 × 10⁵ | 3.04 × 10⁴ | 8.29 |
| MCF7 | NC | 2.60 × 10⁶ | NC |

The expression of the PAI-1 gene was expressed by the ratio of the number of copies of mRNA of the target gene to the number of copies of mRNA of the housekeeping gene. Thus, PAI-1 mRNA was expressed in the MDA-MD-231 cells, whereas it was not detected in the MCF7 cells, in agreement with the expression of these cells.

Table 2 gives the expression of the PAI-1 gene quantified using 50 ng of total RNA originating from ELISA+ tumors, i.e. tumors expressing the PAI-1 protein, or from ELISA− tumors.

TABLE 2
Expression of the PAI-1 gene in ELISA+ and ELISA− tumors

| | Mean of PAI-1 | Mean of PPIB | |
|---|---|---|---|
| | mRNA copies | mRNA copies | PAI-1/PPIB |
| ELISA− tumors | 2.83 × 10⁴ | 1.121 × 10⁶ | 2.5 × 10⁻² |
| ELISA+ tumors | 7.70 × 10⁴ | 1.269 × 10⁶ | 6.1 × 10⁻² |

The expression of the PAI-1 gene was expressed by the ratio of the number of copies of mRNA of the target gene to the number of copies of mRNA of the housekeeping gene. An overexpression of the PAI-1 gene was observed in the ELISA+ tumors.

Based on the PAI-1 and PPIB standard curves previously produced, the detection limit and the quantification limit were determined for the PAI-1 and PPIB genes amplified in duplex. The quantification limit was observed at 1000 copies of mRNA for PAI-1 and PPIB. The detection limit was 100 copies of mRNA for PAI-1 and PPIB.

No signal was observed when the NASBA was carried out using total RNA derived from the PAI-1-negative MCF7 cell line.

All these results were confirmed using another amplification technique (quantitative RT-PCR) (PAI-1: r=0.7886, p<0.0001, n=80; Spearman statistical correlation test). Furthermore, the results obtained at the messenger RNA level by the NASBA technique for the PAI-1 gene correlated with those obtained at the protein level by ELISA (PAI-1: r=0.3590, p=0.0013, n=77; Spearman statistical correlation test). These results demonstrate that the mRNA expression correlates with the presence in the cytosol and the nucleus, confirming the advantage of studying the expression of this gene at the mRNA level.

These results demonstrate that the PAI-1/PPIB duplex by NASBA allows quantification of the expression of the PAI-1 gene from a very small amount of total RNA and, more broadly, from a very small amount of tumor cells, and is entirely suitable for studying the diagnosis/prognosis of breast cancer and the response of a patient to a given treatment.

b2) Duplex Amplification of the UPA and PPIB Genes

This amplification reaction was carried out using a Nuclisens Basic® kit (bioMérieux BV, The Netherlands). For this, 5 ng of total RNA, extracted from the various cell lines, were added to 10 μl NASBA buffer (final concentration in 20 μl of reaction medium: 40 mM of tris HCl, pH 8.5, 12 mM MgCl₂, 70 mM KCl, 5 mM dithiothreito, 15% v/v DMSO, 1 mM of each dNTP, 2 mM of each NTP).

Added to this medium was 0.1 μM of molecular beacons comprising:
SEQ ID No. 10, labeled with a FAM (6-carboxyfluorescein) fluorophore at its 5′ end and a quencher (Dabsyl) at its 3′ end for detecting the RNA encoding UPA at the time of the UPA/cyclophylin B duplex,
SEQ ID No. 15, labeled with a ROX (6-carboxy-X-rhodamine) fluorophore in the 5′ position and a quencher (Dabsyl) in the 3′ position, for detecting the RNA of the PPIB gene.

Also added to this medium were:
0.2 μM of a first UPA primer of SEQ ID No. 5, comprising, at its 5′ end, a sequence comprising the promoter of the T7 polymerase,
0.2 μM of a second UPA primer of SEQ ID No. 6,
0.2 μM of a first PPIB primer of SEQ ID No. 13, comprising, at its 5′ end, a sequence comprising the promoter of the T7 polymerase,
0.2 μM of a second PPIB primer of SEQ ID No. 15.

A preincubation was carried out for 2 minutes at 65° C. before an incubation of 2 minutes at 41° C. A volume of 5 μl of an enzyme mix (0.08 U of RNAse H, 32 U of T7 RNA polymerase, 6.4 U of AMV-RT) was added and an incubation of 90 minutes at 41° C. was carried out.

The quantification of the transcribed RNA was determined in real time according to a principle comparable to that which was described for UPA. The standard curve, as defined previously (dilution transcribed RNA: 10⁸ to 10² copies) was used to quantify the expression of the UPA target gene and of the PPIB housekeeping gene, in order to extrapolate the number of copies of mRNA per sample. The quantification of the expression of a target gene was expressed by a number of copies of mRNA/5 ng of total RNA.

Table 3 gives the expression of the UPA gene quantified using 5 ng of total RNA originating from the MCF-7 and MDA-MD-231 cell lines.

TABLE 3
Expression of the UPA gene in MCF-7 and MDA-MD-231 cells

| | Number of | Number of | |
|---|---|---|---|
| | copies of UPA | copies of PPIB | |
| | mRNA | mRNA | UPA/PPIB |
| MDA-MD-231 | 1.67 × 10⁵ | 2.96 × 10⁵ | 5.62 × 10⁻¹ |
| MCF-7 | NC | 9.49 × 10⁵ | NC |

The expression of the UPA gene was expressed by the ratio of the number of copies of mRNA of the target gene to the number of copies of mRNA of the housekeeping gene. Thus, UPA mRNA was expressed in the MDA321 cells, whereas it was not detected in the MCF-7 cells, in agreement with the expression in these cells.

Table 4 gives the expression of the UPA gene quantified using 50 ng of total RNA originating from ELISA+ tumors or ELISA− tumors.

TABLE 4
Expression of the UPA gene in ELISA+ and ELISA− tumors

| | Mean of copies | Mean of copies | |
|---|---|---|---|
| | of UPA mRNA | of PPIB mRNA | UPA/PPIB |
| ELISA− tumors | 3.70 × 10⁴ | 9.82 × 10⁴ | 3.8 × 10⁻² |
| ELISA+ tumors | 6.70 × 10⁴ | 8.39 × 10⁵ | 8.0 × 10⁻² |

The expression of the UPA gene was expressed by the ratio of the number of copies of mRNA of the target gene to the number of copies of mRNA of the housekeeping gene. An overexpression of the UPA gene was observed in the ELISA+ tumors.

Based on the UPA and PPIB standard curves previously produced, the detection limit and the quantification limit were determined for the UPA and PPIB genes amplified in duplex. The quantification limit was determined at 1000 and 10⁴ copies of mRNA for UPA and PPIB, respectively. The detection limit was 100 copies of mRNA for UPA and PPIB.

Similarly, no signal was observed when the NASBA was carried out using total RNA derived from the UPA-negative MCF7 cell line.

All these results were confirmed using another amplification technique (quantitative RT-PCR) (UPA: r=0.8029, p<0.0001, n=81; Spearman statistical correlation test). Furthermore, the results obtained at the messenger RNA level by the NASBA technique for the UPA gene correlated with those obtained at the protein level by ELISA (UPA: r=0.5784, p<0.0001, n=77; Spearman statistical correlation test). These results demonstrate that the mRNA expression correlates with the presence of proteins in the cytosol, and the nucleus, confirming the advantage of studying the expression of this gene at the mRNA level.

These results demonstrate that the UPA/PPIB duplex by NASBA allows a quantification of the expression of the UPA gene from a very small amount of total RNA and, more broadly, from a very small amount of tumor cells, and is entirely suitable for studying the diagnosis/prognosis of breast cancer and the response of a patient to a given treatment.

1. A method for breast cancer diagnosis/prognosis comprising the following steps:
A—extracting the nucleic material from a biological sample;
B—using at least one pair of amplification primers to obtain amplicons of at least one target sequence of the nucleic material;
C—using at least one detection probe to detect the presence of said amplicons,
wherein, during step B, said pair of primers comprises at least one amplification primer comprising at least 10 nucleotide motifs of a nucleotide sequence selected from SEQ ID NO:1 to SEQ ID NO:8 and/or, during step C, said detection probe comprises at least 10 nucleotide motifs of a nucleotide sequence selected from SEQ ID NO:9 and SEQ ID NO:10.

2. The method for breast cancer diagnosis/prognosis as claimed in claim 1, wherein, during step B, said pair of primers is selected from the following pairs of primers:
a first amplification primer comprising at least 10 nucleotide motifs of the nucleotide sequence SEQ ID NO:1 and a second amplification primer comprising at least 10 nucleotide motifs of the nucleotide sequence SEQ ID NO:2;
a first amplification primer comprising at least 10 nucleotide motifs of the nucleotide sequence SEQ ID NO:3 and a second amplification primer comprising at least 10 nucleotide motifs of the nucleotide sequence SEQ ID NO:4;
a first amplification primer comprising at least 10 nucleotide motifs of the nucleotide sequence SEQ ID NO:5 and a second amplification primer comprising at least 10 nucleotide motifs of the nucleotide sequence SEQ ID NO:6;
a first amplification primer comprising at least 10 nucleotide motifs of the nucleotide sequence SEQ ID NO:7 and a second amplification primer comprising at least 10 nucleotide motifs of the nucleotide sequence SEQ ID NO:8.

3. The method for breast cancer diagnosis/prognosis as claimed in claim 1, in which said pair of primers comprises at least one amplification primer comprising a promoter for the initiation of transcription with a T7 bacteriophage polymerase.

4. The method for breast cancer diagnosis/prognosis as claimed in claim 1, in which, during step C, the detection probe comprises a fluorophore and a quencher.

5. The method as claimed in claim 1, in which the target sequence is included in a gene selected from PAI-1 and UPA-1.

6. The method as claimed in claim 1, in which steps B and C are carried out at the same time.

7. The method as claimed in claim 1, wherein, during step B, at least one pair of amplification primers is also used to obtain amplicons specific for a housekeeping gene.

8. An amplification primer comprising at least 10 nucleotide motifs of a nucleotide sequence selected from SEQ ID NO:1 to SEQ ID NO:8.

9. The amplification primer as claimed in claim 8, further comprising a promoter for the initiation of transcription with a T7 bacteriophage polymerase.

10. A pair of amplification primers selected from the following pairs of primers:
a first amplification primer comprising at least 10 nucleotide motifs of the nucleotide sequence SEQ ID NO:1 and a second amplification primer comprising at least 10 nucleotide motifs of the nucleotide sequence SEQ ID NO:2;
a first amplification primer comprising at least 10 nucleotide motifs of the nucleotide sequence SEQ ID NO:3 and a second amplification primer comprising at least 10 nucleotide motifs of the nucleotide sequence SEQ ID NO:4;
a first amplification primer comprising at least 10 nucleotide motifs of the nucleotide sequence SEQ ID NO:5 and a second amplification primer comprising at least 10 nucleotide motifs of the nucleotide sequence SEQ ID NO:6;
a first amplification primer comprising at least 10 nucleotide motifs of the nucleotide sequence SEQ ID NO:7 and a second amplification primer comprising at least 10 nucleotide motifs of the nucleotide sequence SEQ ID NO:8.

11. The pair of primers as claimed in claim 10, in which said first primer further comprises a promoter for the initiation of transcription with a T7 bacteriophage polymerase.

12. A method for amplifying a target nucleic acid, comprising:
conducting a NASBA amplification reaction with at least one amplification primer according to claim 8.

13. A detection probe comprising at least 10 nucleotide motifs of a nucleotide sequence selected from SEQ ID NO:9 and SEQ ID NO:10.

14. The detection probe as claimed in claim 13, further comprising a fluorophore and a quencher.

15. (canceled)

16. A kit for the diagnosis/prognosis of breast cancer, comprising at least one primer as claimed in claim 8.

17. The method according to claim 12, wherein the amplification primer further comprises a promoter for the initiation of transcription with a T7 bacteriophage polymerase.

18. A method for amplifying a target gene, comprising:
conducting a NASBA amplification reaction with a pair of amplification primers according to claim 10.

19. The method according to claim 18, wherein each amplification primer further comprises a promoter for the initiation of transcription with a T7 bacteriophage polymerase.

20. The kit according to claim 16, wherein each amplification primer further comprises a promoter for the initiation of transcription with a T7 bacteriophage polymerase.

21. The kit according to claim 16, further comprising at least one detection probe comprising at least 10 nucleotide motifs of a nucleotide sequence selected from SEQ ID NO:9 and SEQ ID NO:10.
